(19) United States
(12) Patent Application Publication
(10) Pub. No.: US 2021/0307434 A1
(43) Pub. Date: Oct. 7, 2021

(54) Gloves Designed for Treatment of PTSD, Arthritis, and Other Hand/Wrist Pain

(71) Applicant: Adam Graves, Big Spring, TX (US)

(72) Inventor: Adam Graves, Big Spring, TX (US)

(21) Appl. No.: 16/913,177

(22) Filed: Jun. 26, 2020

Related U.S. Application Data
(60) Provisional application No. 63/005,573, filed on Apr. 6, 2020.

(51) Int. Cl. A41D 19/00; A41D 19/015

(52) U.S. Cl. CPC A41D 19/0037; A41D 19/01552; A41D 19/002; A41D 2500/20; A41D 2500/30; A41D 2500/50; A41D 2400/32

(57) ABSTRACT

This present patent application discloses a glove designed to facilitate comfort while preventing fist clenching, arthritic pain, and hand cramps, and secures conveniently to the hand and wrist via adjustable hook and loop fastener straps. The invention also features a netting or attachment area integrated into a portion of the glove which is capable of supporting and holding therapeutic aids while the user is asleep or to help involuntary muscle contractions. The netting area allows the wearer to squeeze the therapeutic aid if needed, and prevents the user from clenching their hand into a fist while sleeping, minimizing the effects of PTSD, arthritis, etc. in the hands and joints.

### CROSS-REFERENCE TO RELATED APPLICATION

The present application claims priority to, and the benefit of, U.S. Provisional Application No. 63/005,573, which was filed on Apr. 6, 2020 and is incorporated herein by reference in its entirety.

### BACKGROUND

The present invention relates generally to the field of therapeutic gloves. More specifically, the present invention relates to a pair of comfortable gloves, made from fleece or other relaxing or pleasant material and designed to treat or otherwise alleviate post-traumatic stress disorder (“PTSD”), arthritis, and other hand/wrist pain, and thereby prevent a user from clenching his or her hand into a fist while sleeping which may further exacerbate the issue. As used herein, the term “PTSD Gloves” refers to the various kinds of comfortable gloves known variously as Post-traumatic Stress Disorder gloves, Physiotherapy gloves, Trauma gloves, Anti-trauma combat gloves and the like. Accordingly, the present specification makes specific reference thereto and it should be appreciated that aspects of the present invention are also equally amenable to other like applications, devices and methods of manufacture.

Post-traumatic stress disorder, or PTSD as it is commonly referred to, is a mental health condition generally triggered by experiencing or witnessing a terrifying or horrible event. Symptoms of post-traumatic stress disorder may include flashbacks, nightmares, and severe anxiety, as well as uncontrollable or recurring thoughts about the event. Most of the time, people who have PTSD may have temporary difficulty in adjusting and coping, but with time and good self-care, they usually get better and the recall instances lessen if not completely abate. Sometimes, the condition of trauma may last for weeks, months or even years, commonly created by triggers or other occurrences that can bring back memories of the trauma or event accompanied by intense emotional and physical reactions. If the symptoms get worse they can last for months or even years, and the disorder may interfere with PTSD patient's day-to-day life. Also, it is quite common that people with PTSD may inadvertently clench their hands into fists during an episode. Such fist clenching may lead to additional pain, suffering and injury to the patients.

Further, other pain inducing diseases such as arthritis or arthritic affliction, which is a joint inflammation, describes various conditions that affect joints, tissues that surrounds the joint, and other connective tissues. Arthritis is a very common disease and millions of people are diagnosed each year with the ailment. The disease can last for years or may be lifelong, with various treatment regimens required for mitigation of the pain. The disease may affect one or more joints and is generally a swelling or tenderness in the area of the joint. One of the most common types of the disease is osteoarthritis which involves wear-and-tear damage to the joint's cartilage it also affects the entire joint. Osteoarthritis causes changes in the bones and deterioration of the connective tissues that attach muscle to bone and hold the joint together. It also causes inflammation of the joint lining. Another common arthritic disease is rheumatoid arthritis, where the body's immune system attacks the lining of the joint capsule. The lining is a membrane that encloses all the parts of the joint. This lining (synovial membrane) becomes inflamed and swollen and the disease process can eventually destroy cartilage and bone within the joint.

The symptoms of arthritis disorder in a patient usually develop over time but may also appear suddenly in few cases. Arthritis is most seen in adults over the age of 60, but can also develop in children, teens, and younger adults. The joint disorder is more common in women than men and in people who are overweight. People suffering from arthritis disorder or disease may have extreme joint pain and cramps, that persuade a patient to clench their hands into fists while sleeping, and therefore unknowingly leading to further aches, pains, and injury.

Another disorder which may cause clenching of hand is the Clenched Fist Syndrome (“CFS”), which is a type of a psychiatric disorder, in which the patients show flexion finger contractures. Although no organic etiology can be identified, the syndrome in most cases presents with pain and paradoxical stiffness.

By the way of background, the patients suffering from disorders such as PTSD, Arthritis, CFS, and the like, usually consult a doctor and take medicines for the required disorder. The medicines reduce pain and aches due to the disorder and, based on the dosage, the medicines may be effective for a period of time, for example, 3 hours, 6 hours and the like. However, once the effective duration of the medicine is over, the patient again likely suffers extreme pain, cramps, etc. and therefore, inadvertently fist clenching becomes unavoidable until additional medication provides some alleviation of the symptoms.

Another existing solution to combat pain and/or trauma due to pain inducing disorders or diseases is a use of stress ball or a hand exercise ball. Stress balls are made of a squeezable or flexible material and may be molded into any shapes such as spheres, ovals, squares, or even shapes of figures or other designs, etc. Stress balls are portable and an excellent way to relieve stress anywhere. These stress balls are typically squeezed in a hand by a user and are manipulated by fingers, ostensibly to relieve stress and muscle tension or to exercise the muscles of the hand. Stress balls may be made of different materials ranging from soft foam which is highly pliable to more rigid materials which require greater force to use or squeeze. However, the user may utilize such stress relieving balls while awake, as one is unable to hold the ball when sleeping. In case the user suffers from pain due to disorder while sleeping, one has to wake up to hold and use the stress balls. While sleeping, the users generally lack the ability to hold onto the stress balls to alleviate their hand and wrist pain.

Further, gloves exist in the state of the art which are made up of thick material to reduce the effect of fist clenching. However, such hand protection gloves fail to completely minimize the effects of pain inducing disorders and are ineffective with excessive wearing of the gloves by the users. Additionally, some gloves are uniquely designed to treat arthritis pain and feature open fingertip design(s) to provide compression on the fingers and hand to relieve and reduce muscle pain, tendon pain, aching joints due to arthritis, carpal tunnel and swelling. However, such gloves are usually effective only when the user is awake and are not at all useful while sleeping. Another solution has been to roll up a small towel, such as a washcloth and hold the rolled towel in the hand to prevent clenching. However, like a stress ball, the towel may fall out of one's hand when sleeping thereby reducing the effectiveness of the solution.

Therefore, there exists a long felt need in the art for therapeutic gloves that facilitate comfort while preventing clenching, arthritic pain, and hand cramps. There is also a long felt need in the art for gloves that secure conveniently to user's hand and wrist and remain comfortable while wearing. There is a further need for a novel therapeutic device that can be worn on the hands for long periods of time, such as through the sleep cycle. Additionally, there is a long felt need in the art for providing users with gloves that includes mechanisms for supporting stress balls, or other treatment aids, thereby allowing the wearer to squeeze the stress balls if needed. Further, there is a long felt need in the art for gloves for combating pain and trauma due to pain inducing disorders such as PTSD, arthritis, Clenched Fist Syndrome, and the like. Finally, there is a long felt need in the art for gloves which prevent the user from clenching their hand into a fist while sleeping, and therefore minimizing the effects of PTSD, arthritis, etc. in the joints of the hands.

The subject matter disclosed and claimed herein, in one embodiment thereof, comprises a pair of gloves manufactured from a soft, pleasing material and designed to facilitate comfort while preventing or reducing fist clenching, arthritic pain, and hand cramps, and the like. The gloves secure conveniently to user's hand and wrist via an adjustable fastening means, such as a hook and loop fastener strap mechanism, sold under the tradename VELCRO®, although similar products are also available. The invention features a netting or mesh area integrated into a portion of the glove and preferably in the palm portion of the gloves. Alternatively, the mesh area can be in the areas of the fingers or in only one or more of the fingers so that when the fingers close toward the palm they do not unnecessarily splay out of alignment. The netting area is capable of supporting or holding stress balls, and allows the wearer to squeeze the stress balls if needed, and prevents the user from clenching their hand into a fist while sleeping, thereby minimizing the effects of PTSD, arthritis, and other pain inducing disorders on the patients.

In this manner, the pair of gloves of the present invention accomplishes all of the forgoing objectives, and provides a relatively easy and secure solution that is comfortable for the users, easy to wear and prevents clenching, arthritic pain, and hand cramps caused due to pain inducing diseases. In addition, by making the gloves of a washable material, the gloves can be periodically laundered by the user or the care giver. The unique design of the gloves includes a netting or mesh around a portion of the gloves, such as the palm or on one or more of the fingers to support and hold stress balls for relieving stress and pain. The improved pair of gloves of the present invention ensures that the gloves are properly, comfortably secured to the hand and/or wrist via a fastening mechanism such as an adjustable hook and loop fastener straps, snaps, magnets or the like. Finally, the improved gloves of the present invention can be used repeatedly for minimizing the effects of PTSD, arthritis, etc. while sleeping and minimizing the discomfort of the user.

### SUMMARY

The following presents a simplified summary in order to provide a basic understanding of some aspects of the disclosed innovation. This summary is not an extensive overview, and it is not intended to identify key/critical elements or to delineate the scope thereof. The sole purpose is to present some concepts in a simplified form as a prelude to the more detailed description that is presented later and to provide an introductory explanation of the invention.

The subject matter disclosed and claimed herein, in one embodiment thereof, comprises a pair of gloves designed to facilitate comfort while preventing clenching, arthritic pain, and hand cramps, etc. caused due to pain inducing disorders or diseases such as post-traumatic stress disorder, arthritis, Clenched Fist Syndrome, and the like. The gloves secure conveniently to the user's hand and wrist via adjustable hook and loop fastener strap mechanism or other commonly used fastening mechanism, e.g. buttons, snaps, magnets or the like. When magnets are used, they may provide some other therapeutic effects. The fastening mechanism is normally permanently affixed to the gloves and may be sewn into the structure. The invention features a netting, mesh or other attachment area integrated into a palm or other portion of the gloves, which is capable of supporting and holding stress balls, or other therapeutic aids thereby allowing the wearer to squeeze the stress balls or aids if needed. The unique design of the gloves enables a user to hold the stress balls or aids even while sleeping. The gloves prevent the user from clenching their hand into a fist while sleeping, therefore minimizing the effects of PTSD, arthritis, etc. in the hands and on the joints. The mesh or attachment area is wide enough and can hold one or more stress balls or therapeutic aids together inside the mesh or attachment area over palm portion of the gloves. The stress balls or therapeutic aids may be provided with fasteners such as hook and loop type fasteners, snaps, etc. to secure the therapeutic aids to the attachment area. This enables the user to properly grip the stress balls and further press them. The pressing of stress balls relieves the stress and helps prevent or reduce any pain which may be caused due to fist clenching that one may encounter in the morning or when awoken at night due to the discomfort. The attachment or mesh area may be removable from the glove so that the glove could be worn as a normal glove or as a fashion accessory to provide the user with additional options.

The PTSD Gloves described include a pair of comfortable fleece gloves designed to combat PTSD, arthritis, and other hand/wrist pain. The gloves can be slipped on over the hands and secured with an adjustable hook and loop fastener strap or other suitable fastener. Other fastening mechanisms may also be used to secure and adjust the gloves to the size of a user's hands. A central palm area of the gloves features a mesh screen or attachment area, including a pocket or other receptacle capable of receiving one or more stress balls or therapeutic aids. The unique design of the gloves prevents the user from clenching their hand into a fist, thereby preventing additional pain and discomfort in the hands and joints. When the gloves are worn, the stress balls inside the mesh screen around a palm area function as a barrier between the fingers and palm in order to avert fist clenching. The users can squeeze the stress balls as needed for relief. In addition, the attachment area may be on one or more of the fingers such that when the natural tendency to clench a fist occurs, the attachment area on the fingers prevents the fingers from splaying out of a normal position, that is they do not curve outwardly due to swelling of the joint.

In an embodiment, the gloves comprise a netting area around a palm region of the gloves, which are sized and configured and are capable of supporting and holding the stress balls or therapeutic aids. The netting area may hold one or more stress balls together. Further, the netting area may be a detachable mesh piece which can be detached to hold the stress balls and then can be reattached to the palm or other region of the glove through hook and loop fastening mechanisms. Further, other fastening mechanisms may also be used to attach the netting area to the gloves.

In an embodiment, the PTSD gloves include a netting area around an anterior or palm region of the gloves, are capable of supporting and holding stress balls or therapeutic aids. The netting or mesh area piece is attached to an anterior region or a palm region of the gloves. The mesh area includes a cut-out portion, forming a pocket or other receptacle such that the stress balls are inserted inside the mesh area from the cut-out portion. The pocket or receptacle can further be sized and configured to support individual therapeutic aids such that treatment can be applied to individual areas of the hand. For example, each finger could have a pocket or the left and right side of the palm can have a pocket. The cut-out portion can be closed using any fastening mechanisms such as zipper, hook and loop, snaps, buttons, magnets and other fastening methods known in the art.

In an additional embodiment of the presently described invention, includes a treatment glove, having a comfortable material formed into a glove to accommodate a human hand. The glove has an attachment area disposed over a portion of the glove and a fastening attachment for securing the glove to a wrist of a wearer. A therapeutic aid is connected to the attachment area.

In a further embodiment of the present invention, a method of using a therapeutic glove is described and includes the steps of initially providing a glove, the glove having a covering for a hand, palm, thumb and fingers. The glove is secured to the hand by a fastener and the glove has an attachment area connected to the glove. The attachment area is sized and configured to receive at least one therapeutic aid. Next, the hand is inserted into the glove and the glove is secured to the hand. The at least one therapeutic aid is attached to the attachment area, and the therapeutic aid is positioned to reduce formation of a fist by the hand.

In a still further embodiment of the present invention, a pair of therapeutic gloves, is described and includes first and second gloves each manufactured from a comfortable material and each having hand, palm, finger and thumb portions. Each of the first and second gloves has an attachment area for securing therapeutic aids to each of the first and second gloves and for holding the therapeutic aids in a desired location. At least two therapeutic aids each formed from a malleable or pliable material and each of the first and second gloves having at least one therapeutic aid.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a conventional fist clenching during pain due to PTSD, arthritis, and other disorders;

FIG. 2 illustrates a front view of one possible embodiment of a glove of the present invention in accordance with the disclosed structure;

FIG. 3 illustrates a rear view of one possible embodiment of a glove of the present invention in accordance with the disclosed structure;

FIG. 4 illustrates a perspective view of one possible embodiment showing close-up view of the glove and stress balls of the present invention in accordance with the disclosed structure;

FIG. 5 illustrates a perspective view of one possible embodiment showing how the stress balls are squeezed within the glove of the present invention in accordance with the disclosed structure; and

FIG. 6 illustrates one possible embodiment of the glove of the present invention in use while the wearer is asleep in accordance with the disclosed structure.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there exists a long felt need in the art for gloves that facilitate comfort and ease of use while preventing clenching, arthritic pain, and hand cramps. There is also a long felt need in the art for gloves that secure conveniently to user's hand and wrist. Additionally, there is a long felt need in the art for providing users with gloves that includes mechanisms for supporting stress balls or other therapeutic aids, thereby allowing the wearer to squeeze the stress balls or aids if needed. Further, there is a long felt need in the art for gloves for combating pain and trauma due to pain inducing disorders such as PTSD, arthritis, Clenched Fist Syndrome, and the like. Finally, there is a long felt need in the art for gloves which prevent the user from clenching their hand into a fist while sleeping, and therefore minimizing the effects of PTSD, arthritis, etc. in the hands and joints and to help prevent other abnormalities.

The innovative product of the present invention features one or more gloves constructed from a comfortable material, such as fleece, natural or synthetic fibers, woven, non-woven materials and combinations thereof designed to facilitate comfort while preventing or reducing clenching, arthritic pain, and hand cramps. The glove or gloves are secured conveniently to the hand and wrist via adjustable hook and loop fastener straps or other suitable fastening mechanisms, clasps, snaps, buttons, magnets or the like. The unique design of the gloves features a netting area integrated into an anterior region such as the palm region or inside one or more of the fingers in order to support and hold one or more stress balls or therapeutic aids, thereby allowing the wearer to squeeze the stress balls or aids if needed, and prevent the user from clenching his or her hand into a fist while sleeping, thereby minimizing the effects of PTSD, arthritis, etc. in the hands and joints. The netting area allows the user to hold one or more stress balls even when the user is asleep, which reduces stress and pain that is inadvertently caused by any disease or disorder during the sleep of the user. In addition, the netting or attachment areas help to allow the hand and fingers to remain in a more normal position while sleeping or when the gloves are in use during the day if the user suffers from more chronic or persistent problems.

The unique gloves of the present invention can be utilized by patients suffering from post-traumatic stress disorder (PTSD), arthritis, clenched fist syndrome and other hand/wrist pain. Additionally, the gloves may be worn by patients suffering from tissue injuries, pain, cartilage damage, and physical impairments to utilize stress balls for physiotherapy and reduce pain and suffering.

In a most preferred embodiment, the PTSD Gloves of the present invention are a pair of comfortable and washable gloves designed to combat PTSD, arthritis, and other hand/wrist pain. The gloves can be slipped on over the hands and secured with an adjustable hook and loop fastener strap or other suitable fastening means. The central palm area or an anterior region of the gloves, such as the fingers, features a mesh screen capable of receiving or holding one or more stress balls or therapeutic aids. The gloves prevent the user from clenching their hand into a fist, thereby reducing additional pain and discomfort inadvertently caused due to pain inducing disorders. When worn, the stress balls or therapeutic aids function as a barrier between the fingers and palm to prevent a tight fist formation. Users can squeeze the stress balls as needed for relieving the stress.

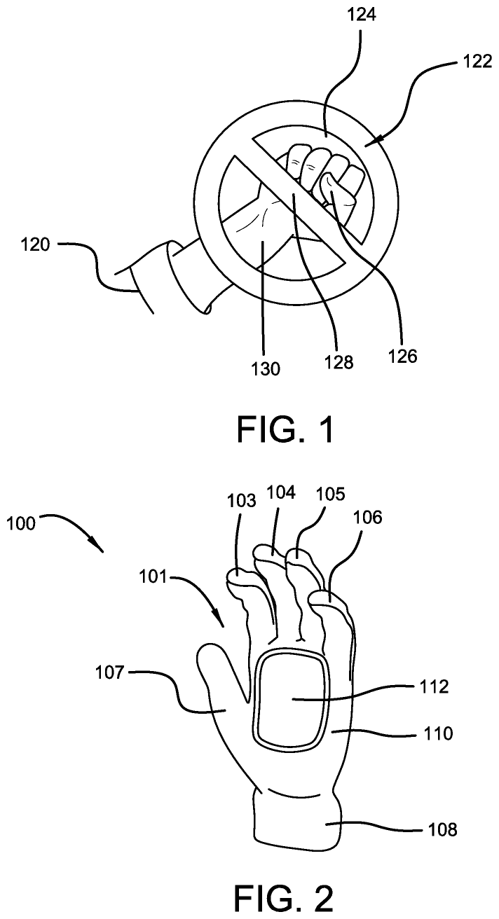

Referring initially to the drawings, FIG. 1 illustrates a depiction of conventional fist clenching during pain due to PTSD, arthritis, and other pain related disorders. Clenched fist syndrome (CFS) and psychoflexed hand are two forms of psychopathological dystonia which result in abnormal hand postures of a user 120. Flexion contraction of the ulnar three digits of the palm is seen in CFS and PTSD and although the thumb and index finger are unaffected, the hand conformation is seen in a fist, thereby resulting in clenched hand 122 condition.

A user folds fingers 124 tightly into a center of a palm 128 and then clamps the thumb 126 over the middle phalanges. This creates an excessive pressure around the palm 128 region, thereby causing additional pain and injury to the hand of the user suffering from the pain inducing disorders. The inadvertent pain caused due to the disorder often adds to the existing pain and stress of the patients. Therefore, a method or a device for preventing fist clenching and combating pain due to disorders such as PTSD, arthritis, etc. is desired in the art.

FIG. 2 illustrates a front view of one possible embodiment of the comfortable fleece gloves 100 of the present invention in accordance with the disclosed architecture. A front portion 101 of the gloves 100 of the present invention comprises a finger section 103, 104, 105, 106; a thumb portion 107, wrist section 108, palm region 110 and a netting, attachment or a mesh area 112. The finger section 103, 104, 105, 106 accommodates all fingers such as index, middle, ring and pinky finger of a user and the thumb portion 107 accommodates a thumb of the user. The wrist section 108 is wrapped around a wrist of the user and is adjusted to a wrist size of the user by utilizing hook and loop fastening strap mechanisms. Alternatively, other known fastening methods may also be utilized to adjust the gloves 100 to user hands, such as clasps, buttons, snaps, magnets or the like. Combinations of fasteners may also be used and when using magnetic fasteners, additional therapeutic benefits may be encountered.

The palm region 110 covers an area excluding fingers and thumb and covers the anterior portion, finger or palm portion of the user hands. The netting, attachment or mesh screen 112 is provided for example over the palm region 110 of the gloves 100. But it should be understood that the area 112 may be positioned elsewhere on the glove to obtain localized treatment benefits such as on individual fingers or the thumb. The mesh screen 112 is made up of net based material and covers an area around anterior portion or palm region 110 of the glove 100. The area 112 may also include fasteners in lieu of a pocket so that therapeutic aids may be attached to the area 112. Further, the mesh screen 112 can be manufactured using any other material such as a fabric or other apparel components, e.g. leather, and the like. The mesh screen 112 expands or is sized and configured to accommodate one or more stress balls or therapeutic aids and therefore allows the user to squeeze the stress balls or aids and reduce stress and trauma caused by disorders such as post-traumatic stress disorder, clenched fist syndrome, arthritis, and the like. The unique design of mesh screen 112 integration over palm region 110 provides an advantage of supporting and holding the stress balls or aids in position while the user is asleep and preventing the balls or aids from dropping from the hand. The stress balls or therapeutic aids may be made from different materials and if multiple aids are used, the individual aids may have differing characteristics such as deformability, hardness, texture or the like.

In a most preferred embodiment, the glove 100 is made by a suitable fleece fabric or an elastic woven material or an elastic fabric. In some cases, the described elastic portion is restricted to portions of palm partly or entirely or fingers, thumb or individual fingers. The attachment area 112 may be positioned on any part of the interior portion of the glove to facilitate the treatment needs of the individual. In further embodiments, a complete glove 100 may be constructed from or comprise various types of compressionable materials such as blends of cotton and elastic materials such as Lycra®. Alternatively, the glove 100 may be made from or comprise various types of compressionable materials further comprising natural or synthetic rubber, synthetic fabrics such as polyester, acrylic, nylon, rayon, acetate, Kevlar®, elastic materials such as elastane fibres, spandex, and Lycra®, and natural fabrics such as coir, cotton, hemp, jute, leather, linen, ramie, wool, silk, or any other suitable flexible natural or synthetic material including combinations or blends of materials. In other embodiments, one or more elements that comprise the glove 100 may be made from durable materials such as hard plastics, metal alloys, wood, hard rubbers, carbon fiber, fiber glass, resins, polymers or any other suitable materials including combinations of materials. Additionally, one or more elements may be made from or comprise durable and slightly flexible materials such as soft plastics, silicone, soft rubbers, fabrics, or any other suitable materials including combinations of materials. Further, the fleece-based glove 100 are soft and provides comfort to the users and are aesthetically appealing.

In an embodiment, the glove 100 is made of an open weave fabric, so that the glove 100 “breathes”, and does not cause the hand to be uncomfortably warm. The glove 100 may also be perforated, so that the hand 120 is not caused to sweat. The gloves are preferably washable or alternatively, may be inexpensive so that they can be disposed of after a specific treatment cycle.

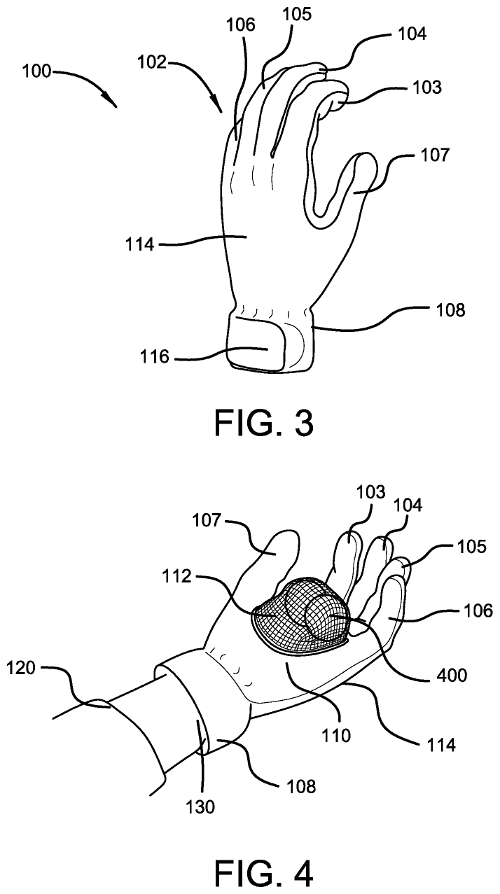

FIG. 3 illustrates a rear view of one possible embodiment of the comfortable therapy gloves 100 of the present invention in accordance with the disclosed structure. The dorsal portion or rear portion 114 of the glove 100 covers a dorsum region or back portion of the user hands. The glove 100 includes a hook and loop fastening strap 116 over the wrist section 108 to secure the glove 100 and adjust the size of the glove 100 to a wrist size of the user's hand. The fastening strap 116 may also be used to provide some additional relief by tightening or loosening the strap as needed. The dorsal portion of the finger sections 103, 104, 105, 106 and thumb section 107 of the glove 100 covers the back portion of the respective fingers and thumb of the user hand.

The adjustable hook 116 is fitted to one end of the wrist section 108 and the hook cross bar is fitted to the opposite end of the wrist section 108. The cross bar fits through a slot in the adjustable hook 116 such that the hook may slide along the length of the cross bar. Moving the adjustable hook 116 along the length of the cross bar allows the wrist section 108 to be fitted to wrists of various sizes, as per the needs of the user. Due to the flexibility and versatility of the wrist section 108, the user may utilize a hook and loop fastening straps 116, for example VELCRO® to fasten wrist section 108 to a size of user's wrist. The fastener helps insure a snug fitting of the glove 100 over the wrist of the user.

FIG. 4 illustrates a perspective view of one possible embodiment showing a close-up view of the glove and therapeutic aids, in this example a pair of deformable stress balls of the present invention in accordance with the disclosed structure. The gloves 100 of the present invention are mainly made to reduce the problems associated with clenching of hands in a PTSD or arthritic patient. As shown in the FIG. 4, the glove 100 is slipped on over a user's hand 120, and different fingers such as index, middle, ring and pinky are accommodated into different finger sections 103, 104, 105 and 106 respectively. Further, a thumb 126 of the user is accommodated into thumb section 107 of the glove 100.

The mesh screen or attachment area 112 over the anterior region or palm region 110 supports and holds the therapeutic aids such as two stress balls 400 inside it, thereby allowing the user to squeeze the stress balls 400, when suffering from inadvertent pain and trauma caused by pain inducing disorders. A stress ball 400 can be made of soft gel, deformable foam or any other elastic material and/or the like which is squeezable by applying pressure to a surface of the stress ball 400. Typically, the stress balls 400 are utilized for relieving stress and are a kind of hand exercise ball. The stress balls 400 may be molded into any shapes such as spheres, ovals, other shape, etc. or other configuration such as may be needed to provide additional comfort or treatment to the individual and may be custom fitted. The therapeutic aids are preferably portable and provide an excellent way to relieve stress any place. The therapeutic aids are generally intended to be inexpensive and replaceable so that they can be discarded if deformed or worn through use over time.

The stress balls 400 are squeezed by a hand of a user and are manipulated by fingers, ostensibly to relieve stress and muscle tension or to exercise the muscles of the hand and potentially forearm. The process of squeezing the stress balls prevents the user from fist clenching and therefore prevents additional pain and injury caused as a consequence of fist clenching. The mesh screen 112 provides advantages to the gloves known in the art by allowing the glove to hold and retain the stress balls 400 while sleeping as well.

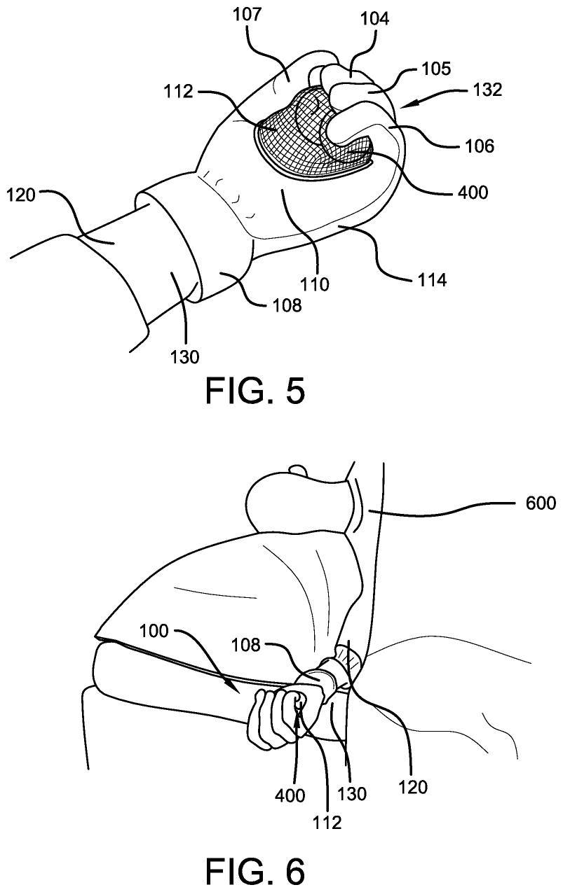

FIG. 5 illustrates a perspective view of one possible embodiment showing how the therapeutic aid in this example, stress balls, are squeezed within the gloves of the present invention in accordance with the disclosed structure. The hand 120 is in a squeezing condition 132 such that fingers tightly grip the stress balls 400 and apply pressure on the balls 400. The stress balls 400 are squeezed by fingers and thumb of the user and are manipulated by fingers, ostensibly to relieve stress and muscle tension or to exercise the muscles of the hand. In an alternate design when the therapeutic aids are only in localized regions, such as for individual fingers, then of course only those localized areas are subject to the treatment or use. The process of squeezing the stress balls prevents the user from fist clenching and therefore prevents additional pain, discomfort and injury caused as a consequence of fist clenching. When the stress balls 400 are placed inside the mesh screen 112 or other attachment area, the stress balls 400 act as a barrier between the fingers and palm to prevent a full fist formation, thereby preventing the fingers from fully contacting the palm region of the hand. Further, one or more balls 400 or therapeutic aids can be inserted inside the mesh screen or pocket 112 of the glove 100.

FIG. 6 illustrates one possible embodiment of the glove of the present invention in use while the wearer is asleep in accordance with the disclosed structure. The pair of gloves 100 of the present invention provides a user 600 with comfort while preventing clenching, and helping to reduce arthritic pain and hand cramps. The glove 100 secures conveniently to the hand 120 and wrist 130 via an adjustable hook and loop fastening straps 108. The gloves 100 of the present invention also feature a netting or attachment area 112 integrated into the anterior portion or palm portion 110 capable of supporting stress balls 400 or other therapeutic aids, thereby allowing the user 600 to squeeze the stress balls 400 when needed, and preventing the user 600 from clenching their hand into the fist while sleeping, minimizing the effects of PTSD, arthritis, etc. in the hands. The mesh screen 112 allows the user 600 to support and hold the stress or hand exercise balls 400 even when the user is asleep. The gloves may also be used by those suffering from muscular or other degenerative diseases where the hand may form into a fist involuntarily.

Further, other stress relieving equipment or devices can also be inserted inside the mesh screen 112, which have similar advantages and effects of stress and pain relief, such as towels, fabric or other compressible or deformable elements. Furthermore, any other device can be utilized, which when placed in the mesh screen 112 prevents fist clenching and the unwanted consequences of fist clenching.

A main advantage of the pair of gloves 100 of the present invention is that the unique and innovative product comprises a mesh screen 112 which forms a pocket or receptacle to hold the stress balls 400 while the user is asleep or as otherwise may be needed to stop involuntary fist formation. The structure of the gloves 100 prevents the user 600 from clenching their hand into a fist, creating additional pain and discomfort in the hands. When the gloves are worn, the stress balls 400 function as a barrier between the fingers and palm to prevent a fist formation. Users 600 may squeeze the stress balls as needed for relief. Thus, it should be apparent that a solution has been provided, in accordance with the present invention, in the form of a glove or a pair of gloves 100 for a user 600 suffering from PTSD, arthritis or other hand cramp related diseases. Users 600 can happily use the pair of gloves 100 of the present invention to reduce discomfort of fist clenching. It can therefore be seen that the instant invention provides a therapeutic support glove which is effective for helping to reduce pain resulting from strain, stress, cramping, swelling and associated pain from the fingers, hands and wrist while performing continuous or repetitive hand clenching. For these reasons, the present invention is believed to represent a significant advancement in the art which has substantial commercial merit.

The therapeutic glove 100 of the present invention comes in pairs or can be manufactured for a single user hand, based on the requirements or medical condition of the user. Further, the gloves 100 may be of any suitable size and color to fulfil the needs of the users. The glove 100 may include a design or pattern as well on the front and back portions to provide additional aesthetic features to the gloves. These designs or patterns could be brand names, teams, logos, seasonal themes or the like.

The term ‘therapeutic gloves’ in the present review is used most broadly and all types of gloves were considered that aim to deliver various rehabilitative treatments for patients: to control and manage hand pain, or to psychologically help to relax or calm the wearer.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “PTSD” and “post-traumatic stress disorder”, are interchangeable and refer to a mental health condition triggered by a terrifying event, causing flashbacks, nightmares and severe anxiety. As used herein “PTSD gloves”, “glove”, “fleece gloves” and “comfortable fleece gloves”, are interchangeable and refer to the pair of fleece gloves 100 of the present invention

As used in the specification, the term “stress ball” refers to a hand exercise ball which is a malleable form, usually not more than 7 cm in diameter, which is squeezed in a hand and manipulated by the fingers, ostensibly to relieve stress and muscle tension or to exercise the muscles of the hand. The malleable form may take any shape or may be custom designed for the particular skeletal or medical requirements of the user.

Notwithstanding the forgoing, the glove or pair of gloves 100 of the present invention and its various structural components can be any suitable size, shape, and configuration as is known in the art without affecting the overall concept of the invention, provided that it accomplishes the above stated objectives. One of ordinary skill in the art will appreciate that the shape and size of the pair of gloves 100 and its various components, as show in the FIGS. are for illustrative purposes only, and that many other shapes and sizes of the pair of gloves 100 are well within the scope of the present disclosure. For example, a partial glove may be constructed covering only one or more of the fingers and a portion of the palm region if localized care is required. Although dimensions of the pair of gloves 100 and its components are important design parameters for good performance, the pair of gloves 100 and its components may be any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A treatment glove, comprising;
a comfortable material formed into a glove to accommodate a human hand, the glove having an attachment area disposed over a portion of the glove;
a fastening attachment for securing the glove to a wrist of a wearer; and
a therapeutic aid connected to the attachment area.

2. A treatment glove as recited in claim 1, wherein the therapeutic aid is a pliable or malleable material;

3. A treatment glove as recited in claim 2, wherein the pliable or malleable material one of a stress ball, towel, foam roll, finger pad or combinations thereof.

4. A treatment glove as recited in claim 1, wherein the fastening attachment is one of a hook and loop fastener, snaps, buttons, magnets or combinations thereof.

5. A treatment glove as recited in claim 1, wherein the attachment area is disposed on at least a portion of the glove in an area for a palm of a hand.

6. A treatment glove as recited in claim 1, wherein the attachment area is disposed on at least one portion of the glove in an area for fingers of a hand.

7. A treatment glove as recited in claim 1, wherein the attachment area is sized and configured to securely hold the therapeutic aid.

8. A treatment glove as recited in claim 1, wherein the attachment area is a pocket sized and configured to hold the therapeutic aid.

9. A treatment glove as recited in claim 1, wherein the attachment area is removable from the glove.

10. A treatment glove as recited in claim 1, wherein the glove is constructed from comfortable materials selected from the group of fleece, natural or synthetic fibers, woven and non-woven materials or combinations thereof.

11. A treatment glove as recited in claim 1, wherein the therapeutic aid is custom designed for a user of the glove.

12. A method of using a therapeutic glove, comprising the steps of;
providing a glove, the glove having a covering for a hand, palm, thumb and fingers, the glove being secured to the hand by a fastener and the glove having an attachment area connected to the glove and the attachment area is sized and configured to receive at least one therapeutic aid;
inserting the hand into the glove;
securing the glove to the hand; and
attaching the at least one therapeutic aid to the attachment area, the therapeutic aid being positioned to reduce formation of a fist by the hand.

13. The method as recited in claim 12, wherein the fastener is selected from hook and loop, snaps, buttons, clasps, magnets or combinations thereof.

14. The method as recited in claim 12, wherein the attachment area is connected to the palm of the glove.

15. The method as recited in claim 12, wherein the attachment area is a mesh pocket.

16. The method as recited in claim 15, wherein the therapeutic aids are inserted into the mesh pocket.

17. The method as recited in claim 12, wherein the therapeutic aid is made from a malleable or pliable material.

18. The method as recited in claim 12, wherein the therapeutic aid is a custom designed form.

19. A pair of therapeutic gloves, comprising;
first and second gloves each manufactured from a comfortable material and each having hand, palm, finger and thumb portions;
each of the first and second gloves having an attachment area for securing therapeutic aids to each of the first and second gloves and for holding the therapeutic aids in a desired location; and
at least two therapeutic aids each formed from a malleable or pliable material and each of the first and second gloves having at least one therapeutic aid.

20. A pair of therapeutic gloves as recited in claim 19, wherein the therapeutic aids may have differing characteristics including deformability, hardness, texture or combinations thereof.
